(19) United States
(12) Patent Application Publication
(10) Pub. No.: US 2023/0237657 A1
(43) Pub. Date: Jul. 27, 2023

(54) INFORMATION PROCESSING DEVICE, INFORMATION PROCESSING METHOD, PROGRAM, MODEL GENERATING METHOD, AND TRAINING DATA GENERATING METHOD

(71) Applicant: TERUMO KABUSHIKI KAISHA, Tokyo (JP)

(72) Inventors: Yuki SAKAGUCHI, Isehara-shi, Kanagawa (JP); Hitoshi TAKEUCHI, Machida-shi, Tokyo (JP)

(73) Assignee: TERUMO KABUSHIKI KAISHA, Tokyo (JP)

(21) Appl. No.: 18/192,390

(22) Filed: Mar. 29, 2023

Related U.S. Application Data
(63) Continuation of application No. PCT/JP2021/035327, filed on Sep. 27, 2021.

(30) Foreign Application Priority Data
Sep. 30, 2020 (JP) 2020-164605

(51) Int. Cl. G06T 7/00; G06T 7/10; G06V 10/764

(52) U.S. Cl. CPC G06T 7/0012; G06T 7/10; G06V 10/764; G06T 2207/10072; G06T 2207/30021; G06T 2207/30101

(57) ABSTRACT

An information processing device configured to: acquire a polar coordinate image, which is a medical image expressed in polar coordinates and obtained by imaging a biological lumen with a device configured to be inserted into the biological lumen, the polar coordinate image having a first axis representing an angle and a second axis intersecting the first axis and representing a distance from the device; input the polar coordinate image for a predetermined angle exceeding 360 degrees to a model trained, when the polar coordinate image is input, to output first segment data in which an image region corresponding to a specific object and another image region are classified, and output the first segment data for the predetermined angle; extract the first segment data for 360 degrees from the first segment data for the predetermined angle; and transform the extracted first segment data to second segment data expressed in rectangular coordinates.

### CROSS-REFERENCES TO RELATED APPLICATIONS

This application is a continuation of International Application No. PCT/JP2021/035327 filed on Sep. 27, 2021, which claims priority to Japanese Application No. 2020-164605 filed on Sep. 30, 2020, the entire content of both of which is incorporated herein by reference.

### TECHNOLOGICAL FIELD

The present disclosure generally relates to an information processing device, an information processing method, a program, a model generating method, and a training data generating method.

### BACKGROUND DISCUSSION

Recent progress in artificial intelligence technologies has led to a proposal of a technique for processing a medical image, using artificial intelligence. For example, Japanese Patent Application Publication No. 2020-520005 A discloses a system and the like for predicting a boundary position of a blood vessel lumen or the like from a medical image obtained by imaging a coronary artery, and using a convolution neural network (CNN) that implements segmentation of an input image.

Medical images include image data expressed in a polar coordinate system, such as an intravascular ultrasound (IVUS) image. The medical image, when displayed, is transformed to a rectangular coordinate system (an X-Y coordinate system) that equates to a real space. In this case, there is a problem that an image expressed in rectangular coordinates may contain a discontinuous portion owing to coordinate system transformation.

According to the disclosure in Japanese Patent Application Publication No. 2020-520005 A, segmentation is implemented after transformation of an image expressed in polar coordinates to an image expressed in rectangular coordinates. However, there is a possibility that a boundary position cannot be accurately predicted due to the problem described above.

### SUMMARY

An information processing device, a program, a model generating method, and a training data generating method are disclosed that are capable of suitably predicting an image region corresponding to a specific object, from a medical image.

An information processing device according to one aspect includes: an acquisition unit configured to acquire a polar coordinate image, the polar coordinate image being a medical image expressed in polar coordinates and obtained by imaging a biological lumen with a device configured to be inserted in the biological lumen, the polar coordinate image having a first axis representing an angle and a second axis intersecting the first axis and representing a distance from the device; an output unit configured to input the polar coordinate image for a predetermined angle exceeding 360 degrees to a model trained, when the polar coordinate image is input, to output first segment data in which an image region corresponding to a specific object and another image region are classified, and configured to output the first segment data for the predetermined angle; an extraction unit configured to extract the first segment data for 360 degrees from the first segment data for the predetermined angle; and a transformation unit configured to transform the extracted first segment data to second segment data expressed in rectangular coordinates.

A non-transitory computer-readable medium storing a program according to another aspect, which when executed by a computer, performs processing comprising: acquiring a polar coordinate image, the polar coordinate image being a medical image expressed in polar coordinates and obtained by imaging a biological lumen with a device configured to be inserted in the biological lumen, the polar coordinate image having a first axis representing an angle and a second axis intersecting the first axis and representing a distance from the device; inputting the polar coordinate image for a predetermined angle exceeding 360 degrees to a model trained, when the polar coordinate image is input, to output first segment data in which an image region corresponding to a specific object and another image region are classified, and outputting the first segment data for the predetermined angle; extracting the first segment data for 360 degrees from the first segment data for the predetermined angle; and transforming the extracted first segment data to second segment data expressed in rectangular coordinates.

A model generating method according to a further aspect comprising: acquiring training data obtained by adding, to a tomographic image expressed in rectangular coordinates and obtained by imaging a biological lumen with a device configured to be inserted in the biological lumen, second segment data in which an image region corresponding to a specific object and another region are classified; respectively transforming the tomographic image and the second segment data to a polar coordinate image having a first axis representing an angle and a second axis intersecting the first axis and representing a distance from the device and first segment data; extracting the polar coordinate image and first segment data for a predetermined angle exceeding 360 degrees from the transformed polar coordinate image and first segment data; and generating a model trained, when the polar coordinate image for the predetermined angle is input, to output the first segment data for the predetermined angle, based on the extracted polar coordinate image and first segment data for the predetermined angle.

In one aspect, an image region corresponding to a specific object can be suitably predicted from a medical image.

### BRIEF DESCRIPTION OF THE DRAWINGS

FIG. 1 is an explanatory diagram illustrating a configuration example of a diagnostic imaging system.

FIG. 2 is a block diagram illustrating a configuration example of a server.

FIG. 3 is a block diagram illustrating a configuration example of a diagnostic imaging device.

FIG. 4 is an explanatory diagram showing a relationship between a polar coordinate image and a blood vessel tomographic image.

FIG. 5A is an explanatory diagram concerning discontinuity of an image owing to coordinate system transformation.

FIG. 5B is an explanatory diagram concerning discontinuity of an image owing to coordinate system transformation.

FIG. 6 is an explanatory diagram concerning object identification processing.

FIG. 7 is an explanatory diagram concerning blood vessel tomographic image display processing.

FIG. 8 is an explanatory diagram concerning identification model generation processing.

FIG. 9 is a flowchart illustrating a procedure for the identification model generation processing.

FIG. 10 is a flowchart illustrating a procedure for the object identification processing.

FIG. 11 is a flowchart illustrating a procedure for object identification processing according to a second embodiment.

FIG. 12 is a flowchart illustrating a procedure for object identification processing according to a third embodiment.

### DETAILED DESCRIPTION

Set forth below with reference to the accompanying drawings is a detailed description of embodiments of an information processing device, an information processing method, a program, a model generating method, and a training data generating method.

### First Embodiment

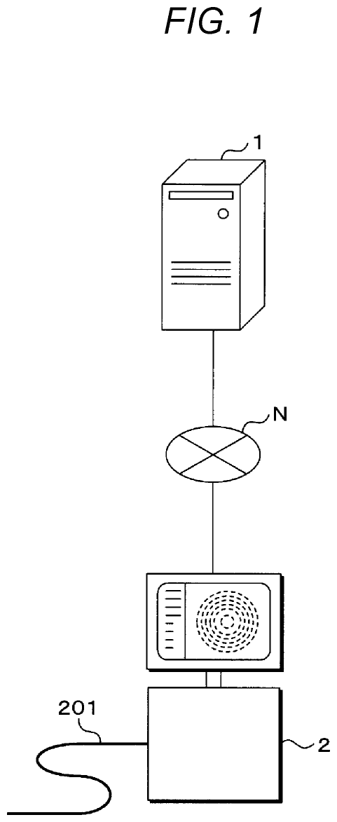

FIG. 1 is an explanatory diagram illustrating a configuration example of a diagnostic imaging system. In a first embodiment, a description will be given of a diagnostic imaging system that predicts, from a medical image obtained by imaging a blood vessel (a biological lumen), an image region corresponding to a specific object in the image (hereinafter, this image region will be referred to as “an object region”). The diagnostic imaging system includes a server 1 and a diagnostic imaging device (an information processing device) 2. The server 1 and the diagnostic imaging device 2 can be communicably connected to each other via a network N such as the Internet.

In the first embodiment, a blood vessel is mentioned as an example of a biological lumen; however, a biological lumen as a subject is not limited to a blood vessel. Examples of the biological lumen may include a bile duct, a pancreatic duct, a bronchus, an intestine, and the like.

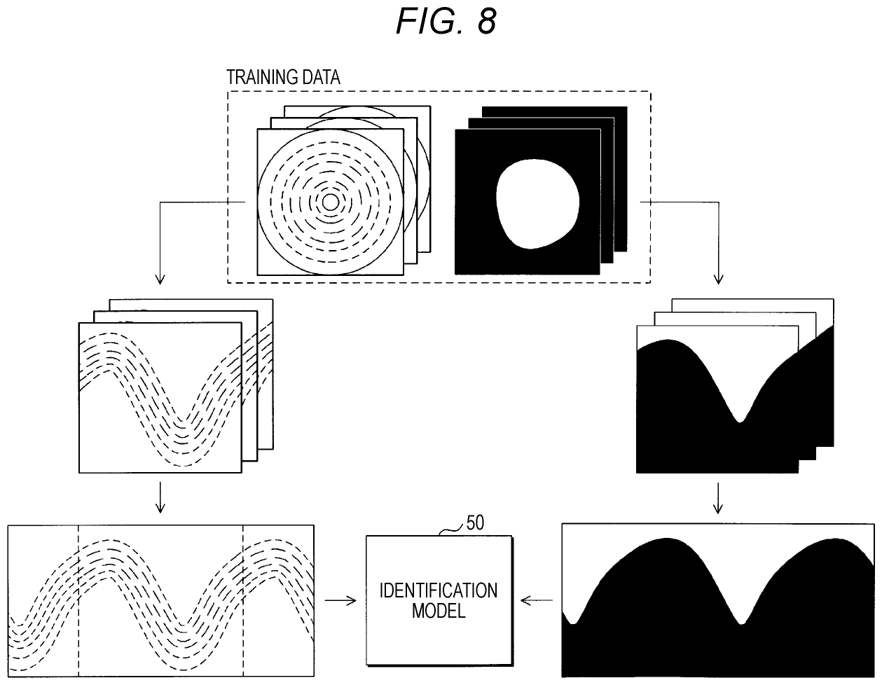

The server 1 is a server computer capable of performing various kinds of information processing, and transmission and reception of information. The server 1 may be, for example, a personal computer or the like. The server 1 functions as a generation device that generates an identification model 50 (see FIG. 8) subjected to machine learning to learn predetermined training data, for identifying an object region, using an input medical image obtained by imaging a blood vessel. As will be described later, the identification model 50 is a machine learning model whose task is to implement segmentation of an image (semantic segmentation of an image region). The identification model 50 outputs segment data in which an object region and another image region in a medical image are classified.

The diagnostic imaging device 2 is an imaging device that captures a medical image by imaging a patient's blood vessel. The diagnostic imaging device 2 can be, for example, an IVUS device that performs an ultrasound inspection using a catheter 201. The catheter 201 is a medical instrument to be inserted into a patient's blood vessel, and an ultrasound probe that transmits and receives an ultrasound signal is mounted to a distal end of the catheter 201. For example, the ultrasound probe is rotatable in a circumferential direction of the catheter 201 and is movable in an axial direction of the blood vessel. The diagnostic imaging device 2 transmits an ultrasound signal from the ultrasound probe, receives a reflected wave, generates an ultrasound tomographic image based on the received reflected wave, and displays the ultrasound tomographic image.

In the first embodiment, an IVUS device is described as an example of the diagnostic imaging device 2. Alternatively, the diagnostic imaging device 2 may be, for example, an optical imaging device employing optical coherence tomography (OCT).

Data on the identification model 50 generated by the server 1 is installed in the diagnostic imaging device 2. The diagnostic imaging device 2 inputs an image captured using the catheter 201 to the identification model 50, and identifies an object region corresponding to a specific object (e.g., an external elastic membrane (EEM) or a lumen). The diagnostic imaging device 2 then displays a blood vessel tomographic image in which the region is identifiable.

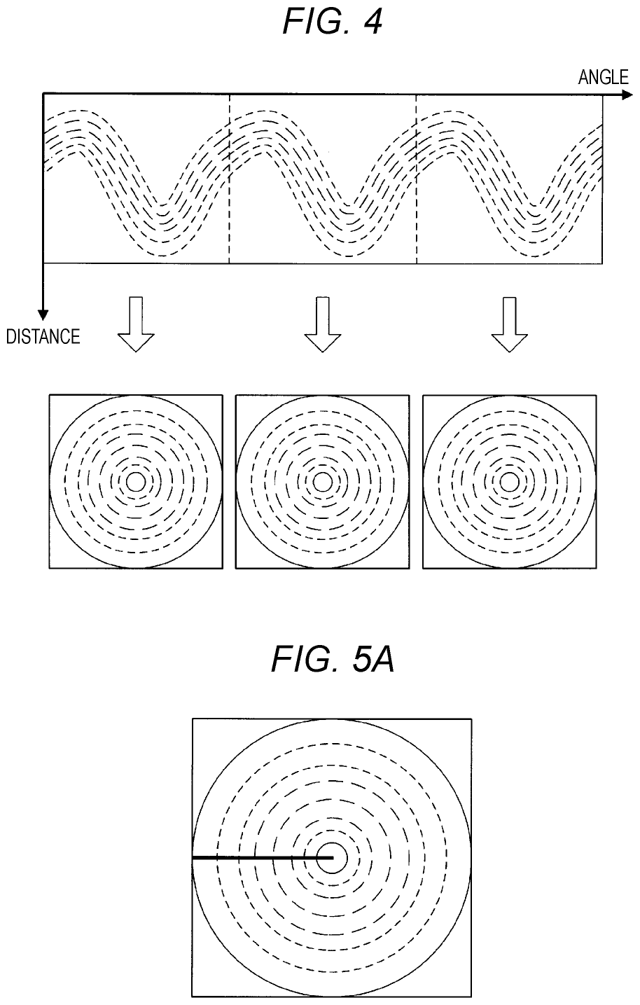

In this case, the diagnostic imaging device 2 identifies the object region from a polar coordinate image having a first axis representing a rotation angle of the ultrasound probe (a device) and a second axis representing a distance from the ultrasound probe. As will be described in detail later, image data to be primarily obtained by the diagnostic imaging device 2 through transmission and reception of an ultrasound signal is not a tomographic image in a rectangular coordinate system to be finally displayed (i.e., a B-mode image), but is an image expressed in polar coordinates (or an array of numerical values) (see FIG. 4). The diagnostic imaging device 2 inputs the polar coordinate image to the identification model 50 and obtains, as an output, segment data in which the object region is classified. The diagnostic imaging device 2 transforms the segment data obtained from the identification model 50 to segment data expressed in rectangular coordinates, and identifies the object region in a final tomographic image.

In the first embodiment, the diagnostic imaging device 2 identifies an object region, using the identification model 50. Alternatively, the server 1 on the cloud may identify an object region. Still alternatively, a general-purpose computer (e.g., a personal computer) connected to the diagnostic imaging device 2 may perform processing. A processing subject that performs the series of processing is not particularly limited.

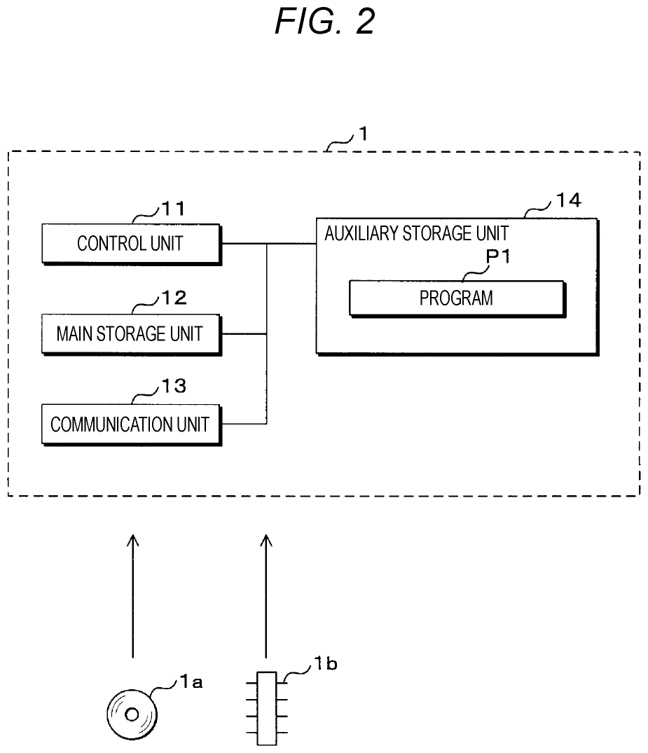

FIG. 2 is a block diagram illustrating a configuration example of the server 1. The server 1 can include a control unit 11, a main storage unit 12, a communication unit 13, and an auxiliary storage unit 14.

The control unit 11 can include one or more arithmetic processing units such as central processing units (CPUs), micro-processing units (MPUs), and graphics processing units (GPUs). The control unit 11 performs various kinds of information processing, control processing, and the like by reading and executing a program P1 stored in the auxiliary storage unit 14. The main storage unit 12 can be a temporary storage region such as a static random access memory (SRAM), a dynamic random access memory (DRAM), or a flash memory. The main storage unit 12 temporarily stores data necessary for the control unit 11 to perform arithmetic processing. The communication unit 13 is a communication module for performing communication-related processing. The communication unit 13 transmits and receives information to and from the outside. The auxiliary storage unit 14 is a nonvolatile memory region such as a large-capacity memory or a hard disk. The auxiliary storage unit 14 stores the program P1 necessary for the control unit 11 to perform processing, and other kinds of data.

The server 1 may be a multi-computer including a plurality of computers, or may be a virtual machine virtually constructed by software.

Further, in the first embodiment, the server 1 is not limited to the above configuration, and may include, for example, an input unit that receives an operation input, a display unit that displays an image, and the like. Further, the server 1 may include a reading unit that reads a portable storage medium 1a such as a compact disk (CD)-ROM or a digital versatile disc (DVD)-ROM, and may execute the program P1 read from the portable storage medium 1a. Alternatively, the server 1 may read the program P1 from a semiconductor memory 1b.

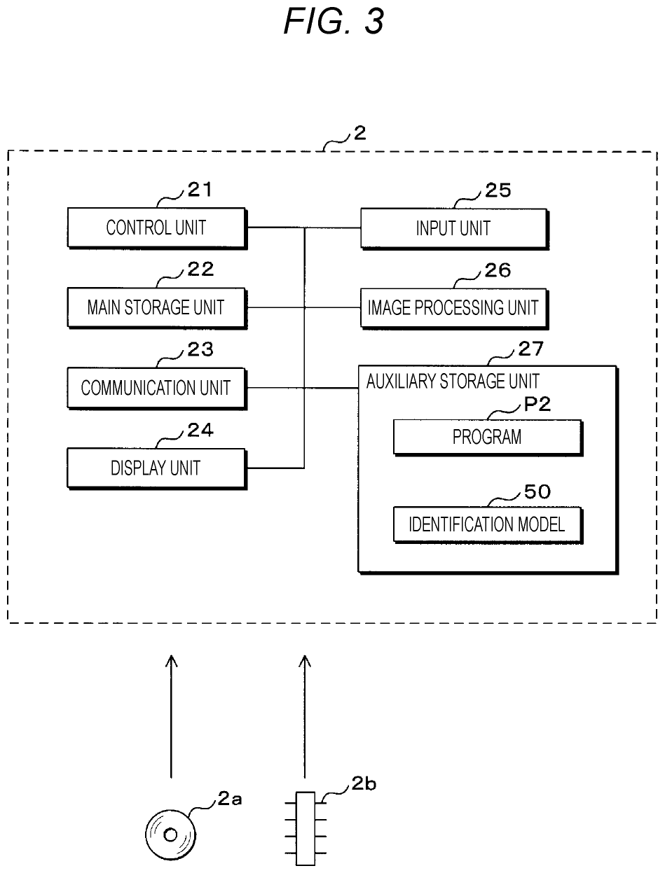

FIG. 3 is a block diagram illustrating a configuration example of the diagnostic imaging device 2. The diagnostic imaging device 2 includes a control unit 21, a main storage unit 22, a communication unit 23, a display unit 24, an input unit 25, an image processing unit 26, and an auxiliary storage unit 27.

The control unit 21 can be, for example, one or more arithmetic processing units such as CPUs, MPUs, and GPUs. The control unit 21 performs various kinds of information processing, control processing, and the like by reading and executing a program P2 stored in the auxiliary storage unit 27. The main storage unit 22 can be a temporary storage region such as a RAM. The main storage unit 22 temporarily stores data necessary for the control unit 21 to perform arithmetic processing. The communication unit 23 is a communication module for performing communication-related processing. The communication unit 23 transmits and receives information to and from the outside. The display unit 24 is a display screen such as a liquid crystal display. The display unit 24 displays an image. The input unit 25 is an operation interface such as a keyboard or a mouse. The input unit 25 receives an operation input from a user. The image processing unit 26 is an image processing module that processes signals transmitted and received via the catheter 201 and generates an image.

The auxiliary storage unit 27 is a nonvolatile memory region such as a hard disk or a large-capacity memory. The auxiliary storage unit 27 stores the program P2 necessary for the control unit 21 to perform processing, and other kinds data. Further, the auxiliary storage unit 27 stores the identification model 50. The identification model 50 is a machine learning model generated by learning with the predetermined training data. Further, the identification model 50 is a model trained to output, with an input polar coordinate image obtained by imaging a blood vessel (a biological lumen), segment data in which an object region and another image region are classified. It is assumed that the identification model 50 is used as a program module constituting a part of artificial intelligence software.

The diagnostic imaging device 2 may include a reading unit that reads a portable storage medium 2a such as a CD-ROM, and may execute the program P2 read from the portable storage medium 2a. Alternatively, the diagnostic imaging device 2 may read the program P2 from a semiconductor memory 2b.

FIG. 4 is an explanatory diagram showing a relationship between a polar coordinate image and a blood vessel tomographic image. FIG. 4 illustrates a state in which a tomographic image for a plurality of frames, expressed in rectangular coordinates, is generated from a polar coordinate image acquired as primary image data.

As described above, the diagnostic imaging device 2 acquires a polar coordinate image as primary image data. The polar coordinate image is image data having a first axis (the horizontal axis in FIG. 4) representing a rotation angle of the ultrasound probe and a second axis (the vertical axis in FIG. 4) representing a distance from the ultrasound probe. Although the first axis and the second axis are orthogonal to each other in FIG. 4, the intersection angle of the first axis and the second axis may be an acute angle or an obtuse angle. In a case where a blood vessel is imaged using the catheter 201, the image is captured in such a manner that the ultrasound probe is rotated and moved in an axial direction of the blood vessel. Therefore, the image thus obtained is primarily expressed in a polar coordinate system. As illustrated in FIG. 4, the diagnostic imaging device 2 extracts an image for 360 degrees (for one rotation) which corresponds to each frame, from the polar coordinate image, transforms the image to a rectangular coordinate system, and generates a blood vessel tomographic image.

In some documents, the image having the first axis representing the angle and the second axis representing the distance (the upper side in FIG. 4) is referred to as “a rectangular coordinate image” and the image resulting from the transformation (the lower side in FIG. 4) is referred to as “a polar coordinate image”. However, in the present specification, the former (i.e., the image having the first axis representing the angle and the second axis representing the distance) is defined as an image in a polar coordinate system, and the latter (i.e., the image resulting from the transformation) is defined as an image in a rectangular coordinate system.

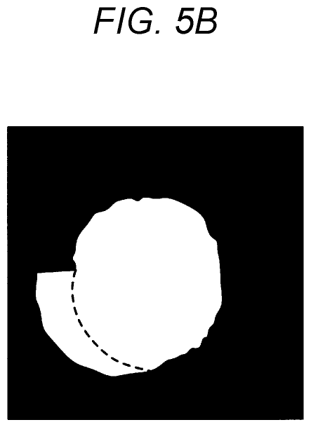

FIGS. 5A and 5B are explanatory diagrams each concerning discontinuity of an image owing to coordinate system transformation. FIG. 5A illustrates a blood vessel tomographic image. FIG. 5B illustrates data (a mask image) obtained by implementing segmentation on the tomographic image. In a case where a polar coordinate image is transformed to a tomographic image expressed in rectangular coordinates, a part of the polar coordinate image continuing along the first axis is extracted and transformed to a tomographic image for each frame. Therefore, portions corresponding to a start point and an end point (0 degrees and 360 degrees) of the rotation angle may become discontinuous. In FIGS. 5A and 5B, a portion at 270 degrees equates to this portion. In a case where segmentation is implemented on the tomographic image in this state, the discontinuous portion may be recognized as it is and a distorted result may be presented.

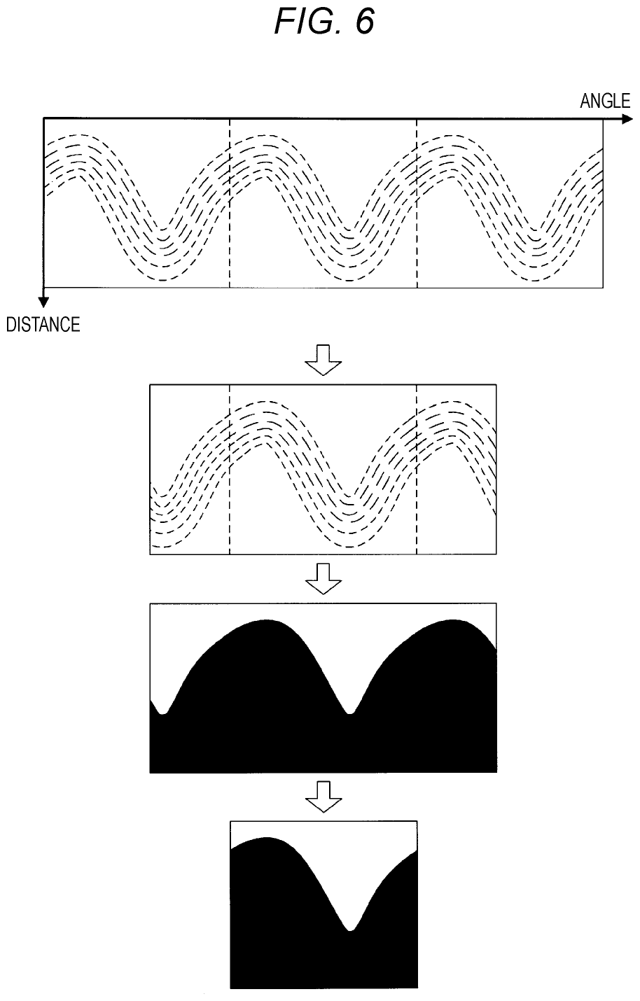

In the first embodiment, therefore, an object region is identified by processing original image data, that is, a polar coordinate image. FIG. 6 is an explanatory diagram concerning object identification processing. FIG. 6 conceptually illustrates a state of identifying an object region from a polar coordinate image.

First, the diagnostic imaging device 2 extracts, from a polar coordinate image captured using the catheter 201, a polar coordinate image corresponding to each frame of a final blood vessel tomographic image. In this case, the diagnostic imaging device 2 extracts a polar coordinate image for a predetermined angle exceeding 360 degrees, rather than a polar coordinate image for one frame, that is, 360 degrees, for each frame of the tomographic image to be finally generated. For example, the diagnostic imaging device 2 extracts a polar coordinate image for 390 degrees including an excess of 15 degrees per frame (360 degrees) added to both end portions of the polar coordinate image for the one frame of 360 degrees along the first axis, and extracts a polar coordinate image for identifying an object region in the tomographic image for each frame.

The excess only needs to exceed by at least a width for one pixel along the first axis and can be optionally designed. Further, the user may optionally set the excess.

The diagnostic imaging device 2 inputs the extracted polar coordinate image to the identification model 50, and identifies the object region. The identification model 50 is a machine learning model generated by learning with predetermined training data. The identification model 50 can be, for example, a semantic segmentation model which is an example of a CNN.

The semantic segmentation model is a neural network that identifies an object in an image on a pixel basis. The semantic segmentation model includes a convolution layer (an encoder) that convolutes an input image and a deconvolution layer (a decoder) that maps a convoluted feature to an original image size. The deconvolution layer identifies a position of an object in an image, based on a feature determined by the convolution layer, and generates binarized data indicating each pixel corresponding to the object.

In the first embodiment, a semantic segmentation model is mentioned as an example of the identification model 50. Alternatively, the identification model 50 may be a model based on another learning algorithm such as a neural network or a generative adversarial network (GAN), in addition to the semantic segmentation model.

The server 1 performs learning on a blood vessel image for training, using training data to which the segment data is added as correct answer data. In the segment data, the object region and the another image region are classified. As a result, the server 1 generates the identification model 50 that outputs, when the polar coordinate image is input, the segment data in which the object region and the another image region are classified. The learning processing concerning the identification model 50 will be described later in detail.

The object region to be identified can be, for example, an EEM region, a lumen region, a region between a lumen boundary and an EEM boundary (i.e., a plaque), or the like in a blood vessel. The identification model 50 according to the first embodiment identifies the image region corresponding to the EEM as the object region. The EEM, the lumen, or the like is an example of the object. For example, the identification model 50 may identify a predetermined device shown in an image (e.g., a guide wire for guiding the catheter 201, a stent indwelled in the blood vessel). Further, the identification model 50 may be capable of simultaneously identifying a plurality of types of objects.

The diagnostic imaging device 2 inputs the polar coordinate image for the predetermined angle extracted as described above to the identification model 50, and obtains, as an output, the segment data for the predetermined angle, in which the object region and the another image region are classified. The segment data is obtained by binarizing the object region and the another image region. In the segment data, a class label indicating a type of region to which each pixel belongs is added to each pixel in the image. FIG. 6 illustrates a mask image in which the object region is indicated with white and the another image region is indicated with black, as an example of the segment data.

The diagnostic imaging device 2 extracts the segment data for 360 degrees that equates to the tomographic image for one frame, from the segment data for the predetermined angle output from the identification model 50. Specifically, as illustrated in FIG. 6, the diagnostic imaging device 2 extracts the segment data for 360 degrees, by removing the segment data for the excess exceeding 360 degrees from both end portions of the segment data for the predetermined angle on the first axis. As a result, segmentation is implemented for the start point and the end point of the rotation angle in consideration of information before the start point and information after the end point (i.e., both the end portions for the excess), so that the object region can be suitably predicted.

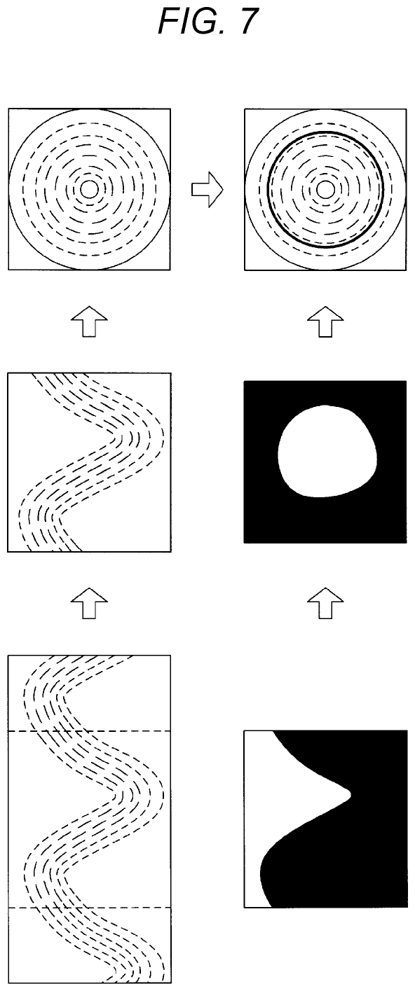

FIG. 7 is an explanatory diagram concerning blood vessel tomographic image display processing. The diagnostic imaging device 2 specifies the object region in the tomographic image expressed in the rectangular coordinates, based on the segment data extracted above. The diagnostic imaging device 2 then generates and displays (outputs) the tomographic image in which the object region is identifiable.

Specifically, the diagnostic imaging device 2 transforms the segment data expressed in the polar coordinate system to the segment data expressed in the rectangular coordinates. Further, the diagnostic imaging device 2 extracts the polar coordinate image for one frame, that is, 360 degrees, from the polar coordinate image input to the identification model 50, that is, the polar coordinate image for the predetermined angle exceeding 360 degrees. The diagnostic imaging device 2 then transforms the extracted polar coordinate image to the rectangular coordinate system and generates the tomographic image.

In the following description, for convenience, the segment data expressed in the polar coordinates (the segment data output from the identification model 50) is referred to as “first segment data”, and the segment data expressed in the rectangular coordinates is referred to as “second segment data”, and both the first segment data and the second segment data are collectively referred to as “segment data” if required.

The diagnostic imaging device 2 generates the tomographic image for output (display) in which the object region is identifiable, based on the second segment data obtained by transforming the first segment data. In the following description, for convenience, the tomographic image for output is referred to as an “output image”. Although a manner to display the output image is not particularly limited, for example, the diagnostic imaging device 2 generates an output image in which a predetermined display object (e.g., a ring-shaped object displayed in color) is superimposed on a boundary between the object region (the EEM region) and the another image region, as illustrated in FIG. 7. In FIG. 7, the display object is indicated by a bold line for convenience of illustration. The diagnostic imaging device 2 displays the generated output image on the display unit 24.

For example, the diagnostic imaging device 2 may output the output image to an external display device (e.g., a monitor or the like installed in a catheter chamber). Further, the diagnostic imaging device 2 may output the output image to a printer or the like, in order to print the output image.

FIG. 8 is an explanatory diagram concerning processing of generating the identification model 50. In the following, a description will be given of machine learning processing for generating the identification model 50.

The server 1 subjects a blood vessel image for training to learning with training data obtained to which correct segment data is added. Here, the blood vessel image for training can be, for example, a tomographic image expressed in rectangular coordinates. For example, the server 1 receives, from a predetermined operator (e.g., a developer of this system), a setting input for adding, to a tomographic image for a plurality of frames, captured in accordance with a pull-back operation of the catheter 201 with regard to an actual patient as a subject, second segment data in which an object region and another image region are classified (e.g., a drawing input for drawing an EEM boundary of a blood vessel). The server 1 generates the identification model 50, using the second segment data set by the operator as correct data.

Since the image and segment data to be input to and output from the identification model 50 are expressed in the polar coordinate system, the server 1 first performs preprocessing of generating training data for transforming the tomographic image for training and the second segment data from the rectangular coordinate system to the polar coordinate system. Specifically, as illustrated in FIG. 8, the server 1 respectively transforms the tomographic image and second segment data for each frame to the polar coordinate image and the first segment data, connects the polar coordinate images and the multiple pieces of first segment data along the first axis, and generates the polar coordinate image and the first segment data corresponding to the plurality of frames. The server 1 extracts a pair of the polar coordinate image and first segment data for the predetermined angle from the generated polar coordinate image and first segment data while shifting a region subject to extraction along the first axis. The server 1 respectively uses the extracted polar coordinate image and first segment data as an input image for training and correct data of an output.

The server 1 performs learning by providing the polar coordinate image and first segment data extracted above to the identification model 50. That is, the server 1 inputs the polar coordinate image to the identification model 50, outputs the first segment data, compares the output first segment data with the correct first segment data, and updates parameters such as a weight between neurons such that the first segment data and the correct first segment data approximate each other. The server 1 performs learning by sequentially providing multiple pairs of the polar coordinate image and the first segment data to the identification model 50 to optimize the parameters. As a result, the server 1 generates the identification model 50.

The above learning method is merely an example; therefore, the first embodiment is not limited to the above learning method. For example, the training data expressed in the polar coordinates (the polar coordinate image and the first segment data) may be provided from the beginning, without the preprocessing of transforming the training data expressed in the rectangular coordinates (the tomographic image and the second segment data) to the polar coordinate system.

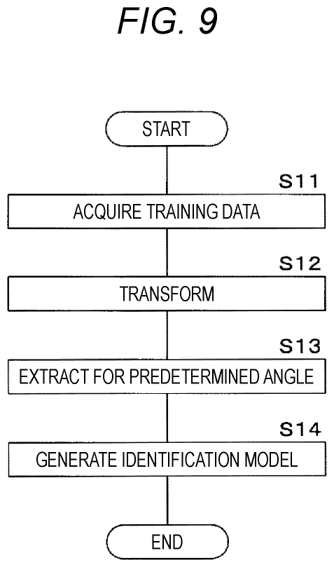

FIG. 9 is a flowchart illustrating a procedure for the processing of generating the identification model 50. With reference to FIG. 9, a description will be given of details of processing in generating the identification model 50 by machine learning.

The control unit 11 of the server 1 acquires training data for generating the identification model 50 (S11). The training data is data obtained by adding, to a blood vessel tomographic image for training, second segment data in which an object region and another image region are classified. The blood vessel tomographic image for training is a blood vessel image expressed in a rectangular coordinates system. Further, the blood vessel tomographic image for training is a tomographic image for a plurality of frames, captured in accordance with a pull-back operation of the catheter 201. The second segment data is data obtained by binarizing the object region and the another image region. Further, the second segment data is segment data expressed in the rectangular coordinate system.

The control unit 11 respectively transforms the tomographic image for training and the second segment data to a polar coordinate image having a first axis representing a rotation angle of the ultrasound probe (the device) and a second axis representing a distance from the ultrasound probe and first segment data coaxial with the polar coordinate image (S12). Specifically, the control unit 11 transforms the tomographic image for each frame to the polar coordinate system, connects the tomographic images along the first axis, and generates the polar coordinate image corresponding to the plurality of frames in the tomographic image expressed in the rectangular coordinates. The control unit 11 also transforms the first segment data corresponding to the tomographic image for each frame to the polar coordinate system, connects the multiple pieces of first segment data along the first axis, and generates the first segment data expressed in the polar coordinates. The control unit 11 respectively extracts the polar coordinate image and first segment data for a predetermined angle exceeding 360 degrees from the transformed polar coordinate image and first segment data (S13).

The control unit 11 generates the identification model 50 that, when the polar coordinate image for the predetermined angle is input, outputs the first segment data in which the object region and the another image region are classified, based on the polar coordinate image and first segment data for the predetermined angle, extracted in S13 (S14). Specifically, the control unit 11 generates a CNN related to semantic segmentation as described above. The control unit 11 inputs the polar coordinate image to the identification model 50, outputs the first segment data, and compares the output first segment data with correct first segment data. The control unit 11 optimizes parameters such as a weight between neurons such that the first segment data and the correct first segment data approximate each other, and generates the identification model 50. The control unit 11 ends the series of processing.

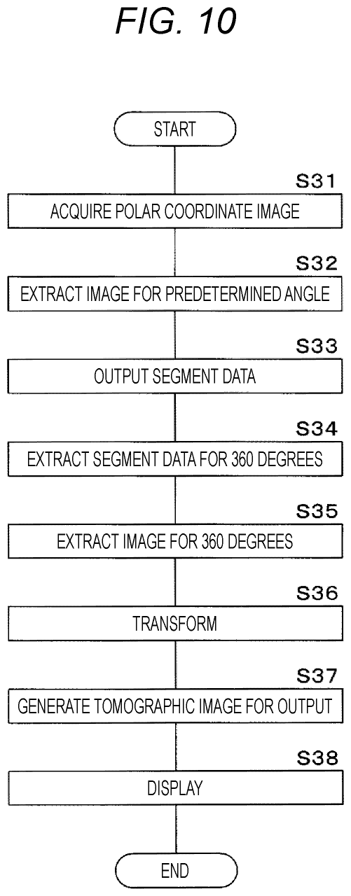

FIG. 10 is a flowchart illustrating a procedure for object identification processing. With reference to FIG. 10, a description will be given of details of processing to be performed by the diagnostic imaging device 2.

The control unit 21 of the diagnostic imaging device 2 acquires a polar coordinate image which is a medical image expressed in the polar coordinates and obtained by imaging a blood vessel (a biological lumen) with the catheter 201 and has a first axis representing a rotation angle of the ultrasound probe (the device) and a second axis representing a distance from the ultrasound probe (S31). The control unit 21 extracts the polar coordinate image for a predetermined angle exceeding 360 degrees from the acquired polar coordinate image (S32).

The control unit 21 inputs the extracted polar coordinate image to the identification model 50, and outputs first segment data in which an object region and another image region are classified (S33). The control unit 21 extracts the first segment data for 360 degrees from the output first segment data (S34). Further, the control unit 21 extracts the polar coordinate image for 360 degrees from the polar coordinate image for the predetermined angle, which is input to the identification model 50 (S35).

The control unit 21 transforms the first segment data extracted in S34 to second segment data expressed in rectangular coordinates, and transforms the polar coordinate image extracted in S35 to a tomographic image expressed in the rectangular coordinates (S36). The control unit 21 generates an output image (a tomographic image) in which the object region is identifiable, based on the transformed second segment data (S37). For example, the control unit 21 generates, as the output image, a tomographic image in which a display object is superimposed on a boundary between the object region (an EEM region) and the another image region. The control unit 21 displays (outputs) the generated output image (S38), and ends the series of processing.

As described above, according to the first embodiment, an object region can be suitably predicted from a medical image expressed in polar coordinates.

Further, according to the first embodiment, an object region predicted from a polar coordinate image can be presented so as to be identifiable in a tomographic image (an output image) expressed in rectangular coordinates.

Further, according to the first embodiment, an object such as an EEM or a lumen serving as a reference for blood vessel image diagnosis can be suitably identified.

Further, according to the first embodiment, data obtained by adding second segment data to a normally observed tomographic image can be transformed to a polar coordinate system and used as training data. As a result, training data creating work (annotation) can be suitably conducted.

### Second Embodiment

In the first embodiment, original image data is used as a polar coordinate image. Alternatively, in a case where original image data is a tomographic image expressed in rectangular coordinates, the tomographic image may be processed by inverse transformation to a polar coordinate system. In a second embodiment, a description will be given of a mode of identifying an object region by inversely transforming a blood vessel tomographic image to a polar coordinate image. Components and steps or processes similar to those described in the first embodiment are denoted with the same reference signs; therefore, the description of the components and steps or processes similar to those described in the first embodiment will not be given.

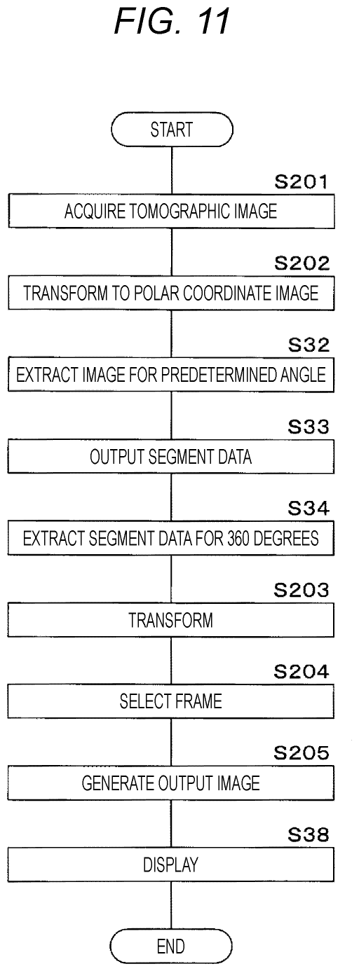

FIG. 11 is a flowchart illustrating a procedure for object identification processing according to the second embodiment. With reference to FIG. 11, a description will be given of details of processing to be performed by a diagnostic imaging device 2 according to the second embodiment.

A control unit 21 of the diagnostic imaging device 2 acquires a tomographic image expressed in rectangular coordinates, which is a medical image obtained by imaging a blood vessel (a biological lumen) (S201). The tomographic image can be, for example, a blood vessel tomographic image captured in the past. Further, the tomographic image can be an image file stored in a format of, for example, digital imaging and communications in medicine (DICOM). For example, the control unit 21 acquires a tomographic image for a plurality of frames, obtained by imaging a blood vessel of a patient who undergone blood vessel treatment or the like in the past, in accordance with a pull-back operation of a catheter 201.

The tomographic image to be processed is not limited to the image file captured in the past, and may be an image captured in real time. Further, the file format of the tomographic image to be processed is not limited to DICOM, and may be any tomographic image expressed in rectangular coordinates.

The control unit 21 transforms the acquired tomographic image to a polar coordinate image (S202). Specifically, the control unit 21 transforms the tomographic image for each frame to a polar coordinate image having a first axis representing an angle and a second axis representing a distance, connects the polar coordinate images along the first axis, and generates the polar coordinate image for the plurality of frames. The control unit 21 causes the processing to proceed to S32.

After extracting the first segment data for 360 degrees from the first segment data for the predetermined angle, which is output from the identification model 50 (S34), the control unit 21 transforms the first segment data to second segment data expressed in the rectangular coordinates (S203). Further, the control unit 21 selects the tomographic image for the frame corresponding to the second segment data from the tomographic image for the plurality of frames acquired in S201 (S204). The control unit 21 generates an output image (a tomographic image) in which an object region is identifiable, based on the second segment data transformed in S203 (S205). The control unit 21 causes the processing to proceed to S38.

As described above, according to the second embodiment, an object region can be suitably predicted also from a tomographic image expressed in rectangular coordinates.

### Third Embodiment

In a third embodiment, a description will be given of a mode of performing relearning (update) on an identification model 50, based on a prediction result of an object region according to the identification model 50.

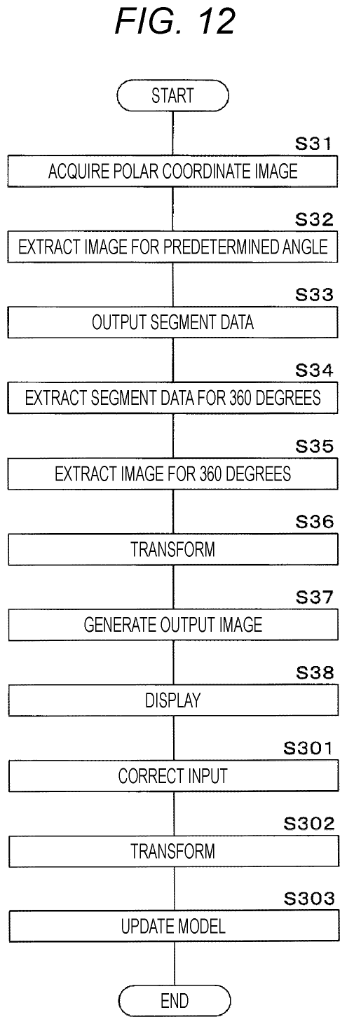

FIG. 12 is a flowchart illustrating a procedure for object identification processing according to the third embodiment. After displaying (outputting) an output image (S38), a diagnostic imaging device 2 performs the following processing.

A control unit 21 of the diagnostic imaging device 2 receives a correction input for correcting an object region indicated in the output image (S301). For example, the control unit 21 receives, with regard to the output image for a plurality of frames, an operation input for redrawing a display object (an EEM boundary) presented as the object region, for example, from a user (a medical worker) who observes the image.

The control unit 21 transforms second segment data representing the corrected object region to first segment data expressed in polar coordinates (S302). Specifically, the control unit 21 inversely transforms the second segment data for each frame to a polar coordinate system, connects the multiple pieces of second segment data along a first axis, and generates the first segment data for the plurality of frames. The control unit 21 updates the identification model 50, based on a polar coordinate image corresponding to the output image (a tomographic image) and the first segment data transformed in S302 (S303). Specifically, the control unit 21 provides the polar coordinate image for a predetermined angle, input to the identification model 50 in S33, and the first segment data for the predetermined angle corresponding to the polar coordinate image among the multiple pieces of first segment data generated in S302, to the identification model 50, as training data for relearning to update parameters such as a weight between neurons. The control unit 21 ends the series of processing.

In the above description, the diagnostic imaging device 2 performs the update (relearning) processing in S303. Alternatively, a server 1 may perform this processing.

As described above, according to the third embodiment, prediction accuracy can be improved in such a manner that relearning is performed based on a prediction result of an object region according to the identification model 50.

The detailed description above describes embodiments of an information processing device, an information processing method, a program, a model generating method, and a training data generating method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An information processing device comprising:
a processor configured to:
acquire a polar coordinate image, the polar coordinate image being a medical image expressed in polar coordinates and obtained by imaging a biological lumen with a device configured to be inserted into the biological lumen, the polar coordinate image having a first axis representing an angle and a second axis intersecting the first axis and representing a distance from the device;
input the polar coordinate image for a predetermined angle exceeding 360 degrees to a model trained, when the polar coordinate image is input, to output first segment data in which an image region corresponding to a specific object and another image region are classified, and configured to output the first segment data for the predetermined angle;
extract the first segment data for 360 degrees from the first segment data for the predetermined angle; and
transform the extracted first segment data to second segment data expressed in rectangular coordinates.

2. The information processing device according to claim 1, wherein the processor is further configured to:
output, based on the second segment data, a tomographic image in which the image region corresponding to the object region is identifiable, the tomographic image obtained by transforming the polar coordinate image to a rectangular coordinate system.

3. The information processing device according to claim 2, wherein the processor is further configured to:
receive a correction input for correcting the image region corresponding to the object, after output of the tomographic image;
transform the second segment data representing the corrected image region corresponding to the object, to the first segment data; and
update the model, based on the polar coordinate image and the transformed first segment data.

4. The information processing device according to claim 1, wherein the polar coordinate image is an image obtained by imaging a blood vessel with a catheter configured to be inserted in the blood vessel.

5. The information processing device according to claim 4, wherein the processor is further configured to:
output the first segment data in which an image region corresponding to an external elastic membrane or a lumen of the blood vessel is classified.

6. The information processing device according to claim 1, wherein the processor is further configured to:
extract the first segment data for 360 degrees by removing the first segment data for an excess exceeding 360 degrees, from both end portions of the first segment data for the predetermined angle.

7. The information processing device according to claim 1, wherein the processor is further configured to:
acquire a tomographic image expressed in rectangular coordinates and obtained by imaging the biological lumen;
transform the tomographic image to the polar coordinate image; and
input the transformed polar coordinate image to the model.

8. The information processing device according to claim 7, wherein the processor is further configured to:
output the first segment data.

9. A non-transitory computer-readable medium storing a program, which when executed by a computer, performs processing comprising:
acquiring a polar coordinate image, the polar coordinate image being a medical image expressed in polar coordinates and obtained by imaging a biological lumen with a device configured to be inserted in the biological lumen, the polar coordinate image having a first axis representing an angle and a second axis intersecting the first axis and representing a distance from the device;
inputting the polar coordinate image for a predetermined angle exceeding 360 degrees to a model trained, when the polar coordinate image is input, to output first segment data in which an image region corresponding to a specific object and another image region are classified, and outputting the first segment data for the predetermined angle;
extracting the first segment data for 360 degrees from the first segment data for the predetermined angle; and
transforming the extracted first segment data to second segment data expressed in rectangular coordinates.

10. The non-transitory computer-readable medium according to claim 9, further comprising:
outputting, based on the second segment data, a tomographic image in which the image region corresponding to the object region is identifiable, the tomographic image obtained by transforming the polar coordinate image to a rectangular coordinate system.

11. The non-transitory computer-readable medium according to claim 10, further comprising:
receiving a correction input for correcting the image region corresponding to the object, after output of the tomographic image;
transforming the second segment data representing the corrected image region corresponding to the object, to the first segment data; and
updating the model, based on the polar coordinate image and the transformed first segment data.

12. The non-transitory computer-readable medium according to claim 9, wherein the polar coordinate image is an image obtained by imaging a blood vessel with a catheter inserted in the blood vessel, and further comprises:
outputting the first segment data in which an image region corresponding to an external elastic membrane or a lumen of the blood vessel is classified.

13. The non-transitory computer-readable medium according to claim 9, further comprising:
extracting the first segment data for 360 degrees by removing the first segment data for an excess exceeding 360 degrees, from both end portions of the first segment data for the predetermined angle.

14. The non-transitory computer-readable medium according to claim 9, further comprising:
acquiring a tomographic image expressed in rectangular coordinates and obtained by imaging the biological lumen, and transform the tomographic image to the polar coordinate image; and
inputting the transformed polar coordinate image to the model, and output the first segment data.

15. A model generating method comprising:
acquiring training data obtained by adding, to a tomographic image expressed in rectangular coordinates and obtained by imaging a biological lumen with a device configured to be inserted in the biological lumen, second segment data in which an image region corresponding to a specific object and another region are classified;
respectively transforming the tomographic image and the second segment data to a polar coordinate image having a first axis representing an angle and a second axis intersecting the first axis and representing a distance from the device and first segment data;
extracting the polar coordinate image and first segment data for a predetermined angle exceeding 360 degrees from the transformed polar coordinate image and first segment data; and
generating a model trained, when the polar coordinate image for the predetermined angle is input, to output the first segment data for the predetermined angle, based on the extracted polar coordinate image and first segment data for the predetermined angle.

16. The model generating method according to claim 15, further comprising:
outputting, based on the second segment data, a tomographic image in which the image region corresponding to the object region is identifiable, the tomographic image obtained by transforming the polar coordinate image to a rectangular coordinate system.

17. The model generating method according to claim 16, further comprising:
receiving a correction input for correcting the image region corresponding to the object, after output of the tomographic image;
transforming the second segment data representing the corrected image region corresponding to the object, to the first segment data; and
updating the model, based on the polar coordinate image and the transformed first segment data.

18. The model generating method according to claim 15, wherein the polar coordinate image is an image obtained by imaging a blood vessel with a catheter inserted in the blood vessel, and further comprises:
outputting the first segment data in which an image region corresponding to an external elastic membrane or a lumen of the blood vessel is classified.

19. The model generating method according to claim 15, further comprising:
extracting the first segment data for 360 degrees by removing the first segment data for an excess exceeding 360 degrees, from both end portions of the first segment data for the predetermined angle.

20. The model generating method according to claim 15, further comprising:
acquiring a tomographic image expressed in rectangular coordinates and obtained by imaging the biological lumen, and transform the tomographic image to the polar coordinate image; and
inputting the transformed polar coordinate image to the model, and output the first segment data.
